(19) United States
(12) Patent Application Publication
(10) Pub. No.: US 2019/0350876 A1
(43) Pub. Date: Nov. 21, 2019

(54) ORAL THIN FILMS COMPRISING PLANT EXTRACTS AND METHODS OF MAKING AND USING SAME

(71) Applicant: F6 Pharma, Inc., Ruxton, MD (US)

(72) Inventors: Dana Carmel Hammell, Georgetown, KY (US); Audra Lynn Stinchcomb, Ruxton, MD (US); Nihar Shah, Ruxton, MD (US)

(21) Appl. No.: 16/461,560

(22) PCT Filed: Nov. 16, 2017
(86) PCT No.: PCT/US2017/062001

§ 371 Date: May 16, 2019

Related U.S. Application Data
(60) Provisional application No. 62/423,258, filed on Nov. 17, 2016.

(51) Int. Cl. A61K 31/05; A61K 9/70; A61K 9/00; A61K 47/38; A61K 47/12; A61K 47/44; A61K 47/22

(52) U.S. Cl. CPC A61K 31/05; A61K 9/7007; A61K 9/0056; A61K 47/22; A61K 47/12; A61K 47/44; A61K 47/38

(57) ABSTRACT

The present disclosure provides compositions comprising cannabidiol and methods of making and using same.

### CROSS-REFERENCE TO RELATED APPLICATION

The present application is a § 371 U.S. National Stage Application of International Application No. PCT/US2017/062001 filed on Nov. 16, 2017, which claims priority to U.S. Provisional Application No. 62/423,258 filed on Nov. 17, 2016, the entirety of each of which is incorporated herein by reference and relied upon.

### TECHNICAL FIELD

The present disclosure relates to compositions (e.g., oral compositions such as an oral thin film) comprising cannabidiol (e.g., purified cannabidiol) and methods of making and using same.

### BACKGROUND

Cannabidiol (“CBD”) is one of over 400 known compounds present in cannabis plants. Although its full range of pharmacological uses is not yet understood, CBD is known to act as a partial agonist of 5-HT_{1A} receptor, as an antagonist of GPR55 receptor, and as an indirect antagonist of CB₁ and CB₂ receptors. CBD is highly hydrophobic, which presents challenges for formulating high purity compositions suitable for convenient administration to human and animal subjects. A need therefore exists for stable and convenient CBD dosage forms.

### SUMMARY

The present disclosure provides compositions (e.g., oral compositions such as an oral thin film) comprising cannabidiol (e.g., purified cannabidiol) and methods of making and using same.

In some embodiments, the present disclosure provides an oral delivery form comprising a cannabis extract, such as cannabidiol.

In some embodiments, the present disclosure provides a unit dosage form comprising a plant extract, wherein the plant extract comprises cannabidiol.

In some embodiments, the present disclosure provides an oral thin film comprising cannabidiol.

### DETAILED DESCRIPTION

The present disclosure provides compositions comprising cannabidiol, optionally in the form of a cannabis extract, and methods of making and using same for treating a disease or disorder in a subject.

Cannabidiol (“CBD”) is a hydrophobic terpenoid produced by cannabis plants. The species of cannabis plants include Cannabis sativa, Cannabis indica, and Cannabis ruderalis. Varieties contain different amounts of CBD, with hemp producing higher concentrations of CBD and lower concentrations of the psychoactive cannabinoid tetrahydrocannabinol (THC). In one embodiment, the CBD is extracted from hemp. CBD is a series of isomers represented by the following general structure

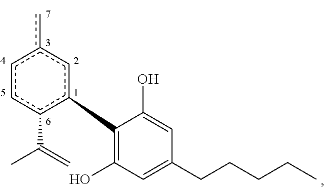

wherein a carbon-carbon double bond is present at one of the indicated

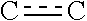

positions of the methy/methylenyl cyclohexenyl ring. The seven positional isomers are summarized below using IUPAC nomenclature corresponding to the general structure shown above.

| Short Name | IUPAC Name | Structure |
|---|---|---|
| Δ^{l}-cannabidiol | 2-(6-isopropenyl-3-methyl-1- cyclohexen-1-yl)-5-pentyl-1,3- benzenediol | 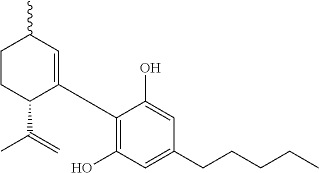 [SMILES: C=C(C)[C@@H]1CCC(C)C=C1Cc1c(O)cc(CCCCC)cc1O] |
| Δ²-cannabidiol | 2-(6-isopropenyl-3-methyl-2- cyclonexen-1-yl)-5-pentyl-1, 3-benzenediol | 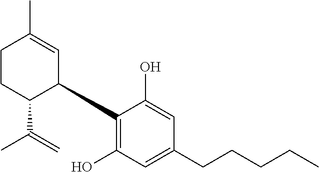 [SMILES: C=C(C)[C@@H]1CCC(C)=C[C@H]1c1c(O)cc(CCCCC)cc1O] |
| Δ³-cannabidiol | 2-(6-isopropenyl-3-methyl-3- cyclohexen-1-yl)-5-pentyl-1 3-benzenediol | 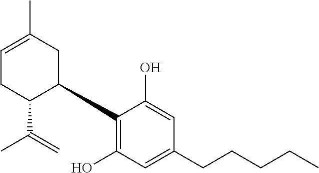 [SMILES: C=C(C)[C@@H]1CC=C(C)C[C@H]1c1c(O)cc(CCCCC)cc1O] |
| Δ^{3,7}-cannabidiol | 2-(6-isopropenyl-3- methylenecyclohex-1-yl)-5- pentyl-1,3-benzenediol | 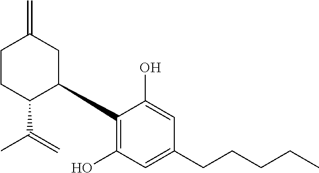 [SMILES: C=C1CC[C@@H](C(=C)C)[C@H](c2c(O)cc(CCCCC)cc2O)C1] |
| Δ⁴-cannabidiol | 2-(6-isopropenyl-3-methyl-4- cyclohexen-1-yl)-5-pentyl-1,3- benzenediol | 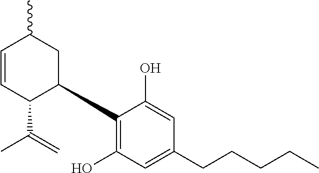 [SMILES: C=C(C)[C@@H]1C=CC(C)C[C@H]1c1c(O)cc(CCCCC)cc1O] |
| Δ⁵-cannabidiol | 2-(6-isopropenyl-3-methyl-5- cyclohexen-1-yl)-5-pentyl-1,3- benzenediol | 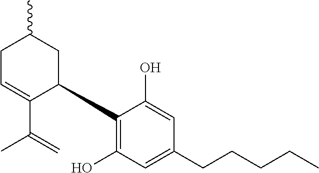 [SMILES: C=C(C)C1=CCC(C)C[C@H]1c1c(O)cc(CCCCC)cc1O] |
| Δ⁶-cannabidiol | 2-(6-isopropenyl-3-methyl-6- cyclohexen-1-yl)-5-pentyl-1,3- benzenediol | 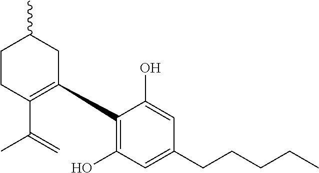 |

In some embodiments, a composition of the present disclosure includes an active agent comprising, consisting essentially of, or consisting of CBD. In some embodiments, the active agent comprises one of the CBD isomers described above. In some embodiments, the active agent comprises a mixture of more than one of the CBD isomers described above, such as two of the isomers, three of the isomers, four of the isomers, five of the isomers, six of the isomers, or all seven of the isomers described above. In some embodiments, the active agent comprises, consists essentially of, or consists of a derivative of any one or more of the CBD isomers described above, such as an O-protected isomer, an ester, or an alkyl ether derivative.

In some embodiments, CBD (e.g., one or more CBD isomers or derivative(s) thereof) represents at least about 80% of all cannabis-related compounds in the composition. In some embodiments, CBD (e.g., one or more CBD isomers or derivative(s) thereof) represents at least about 90% of all cannabis-related compounds in the composition. In some embodiments, CBD represents at least about 95% of all cannabis-related compounds in the composition. In some embodiments, CBD represents at least about 96% of all cannabis-related compounds in the composition. In some embodiments, CBD represents at least about 97% of all cannabis-related compounds in the composition. In some embodiments, CBD represents at least about 98% of all cannabis-related compounds in the composition. In some embodiments, CBD represents at least about 99% of all cannabis-related compounds in the composition.

In some embodiments, an oral thin film composition of the present disclosure comprises one or more matrix polymers. As used herein, the term “matrix polymers” refers generally to high molecular weight (MW) polymers that can form high viscosity solutions in water or a volatile solvent, can dissolve or disperse an active agent, and can be cast as stable, self-standing films. In some embodiments, the matrix polymer comprises one or more of: a polyvinyl caprolactam-polyvinyl acetate-polyethylene glycol graft co-polymer, carboxymethyl cellulose (CMC), hydroxymethyl cellulose (HPC), hydroxypropylmethyl cellulose (HPMC), polyethylene oxide) (PEO), polyvinyl alcohol) (PVA), and poly(acrylic acid). In some embodiments, dispersion of cannabidiol in the matrix polymer is accomplished by first dissolving the cannabidiol (e.g., a cannabis extract comprising cannabidiol) in a small amount of solvent (e.g., ethanol), and the cannabidiol solution is then mixed with the matrix polymer(s). In such embodiments, the matrix polymer(s) should be miscible with the cannabidiol solvent to avoid compromising integrity of the final oral thin film and to avoid unappealing mouth feel from, for example, precipitation of the matrix polymer. In some embodiments, the matrix polymer comprises, consists essentially of, or consists of a polyvinyl caprolactam-polyvinyl acetate-polyethylene glycol graft co-polymer (e.g., Soluplus, BASF Cat. No. 410343-S), a pharmaceutical grade hydroxypropyl cellulose-based polymer (e.g., Klucel JXF, Ashland Cat. No. 420040), or a combination thereof. In some embodiments, the matrix polymer comprises, consists essentially of, or consists of a polyvinyl caprolactam-polyvinyl acetate-polyethylene glycol graft co-polymer. In some embodiments, the matrix polymer comprises, consists essentially of, or consists of a pharmaceutical grade hydroxypropyl cellulose-based polymer.

In some embodiments, an oral thin film composition of the present disclosure comprises one or more plasticizers. Any suitable plasticizer may be used, such as propylene glycol. When present, a plasticizer may represent about 0.5 wt. % to about 20 wt. % of the total weight of the oral thin film composition (e.g., total weight before casting), such as about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, about 15 wt. %, about 15.5 wt. %, about 16 wt. %, about 16.5 wt. %, about 17 wt. %, about 17.5 wt. %, about 18 wt. %, about 18.5 wt. %, about 19 wt. %, about 19.5 wt. %, or about 20 wt. % of the total weight of the oral thin film composition (e.g., total weight before casting).

In some embodiments, a solvent is used to prepare a solution of the matrix polymer, the active agent, and one or more excipients for casting into thin film form. In some embodiments, the solvent comprises ethanol. In some embodiments, the solvent comprises water.

In some embodiments, an oral thin film of the present disclosure comprises one or more excipients. In some embodiments, the one or more excipients comprise a preservative, a flavoring, a sweetener, a colorant, a salivating agent, a penetration enhancer, or a combination thereof.

In some embodiments, the excipient comprises a preservative. In some embodiments, the preservative comprises, consists essentially of, or consists of butylated hydroxytoluene (BHT) (e.g., Spectrum Chemical Cat. No. B1196, New Brunswick, N.J.). In some embodiments, the preservative is present in an amount of about 0.01 wt. % to about 0.5 wt. % of the total weight of the oral thin film composition (e.g., total weight before casting), such as about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, or about 0.5 wt. % of the total weight of the oral thin film composition (e.g., total weight before casting). In some embodiments, the oral thin film comprises substantially no added preservative or no added preservative.

In some embodiments, the excipient comprises a flavorant. In some embodiments, the flavorant comprises, consists essentially of, or consists of peppermint oil (e.g., Mother Murphy's Cat. No. 2318186, Greensboro N.C.). In some embodiments, the flavorant is present in an amount of about 0.2 wt. % to about 2 wt. % of the total weight of the oral thin film composition (e.g., total weight before casting), such as about 0.2 wt. %, about 0.4 wt. %, about 0.6 wt. %, about 0.8 wt. %, about 1 wt. %, about 1.2 wt, %, about 1.4 wt. %, about 1.6 wt. %, about 1.8 wt, %, or about 2 wt. % of the total weight of the oral thin film composition (e.g., total weight before casting). In some embodiments, the oral thin film comprises substantially no added flavorant or no added flavorant.

In some embodiments, the excipient comprises a sweetener. In some embodiments, the sweetener comprises, consists essentially of, or consists of xylitol (e.g., Spectrum Chemical Cat. No. X1017, New Brunswick, N.J.). In some embodiments, the sweetener is present in an amount of about 0.2 wt. % to about 2 wt. % of the total weight of the oral thin film composition (e.g., total weight before casting), such as about 0.2 wt. %, about 0.4 wt. %, about 0.6 wt. %, about 0.8 wt. %, about 1 wt. %, about 1.2 wt. %, about 1.4 wt. %, about 1.6 wt. %, about 1.8 wt. %, or about 2 wt. % of the total weight of the oral thin film composition (e.g., total weight before casting). In some embodiments, the oral thin film comprises substantially no added sweetener or no added sweetener.

In some embodiments, the excipient comprises a colorant. In some embodiments, the colorant comprises, consists essentially of, or consists of chlorophyll (e.g., Swanson Health Products Cat. No. DES001, Fargo, N. Dak.). In some embodiments, the colorant is present in an amount of about 0.1 wt. % to about 1 wt. % (e.g., total weight before casting), such as about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, or about 1 wt. % (e.g., total weight before casting). In some embodiments, the oral thin film comprises substantially no added colorant or no added colorant.

In some embodiments, the excipient comprises a salivating agent. In some embodiments, the salivating agent comprises, consists essentially of, or consists of citric acid (e.g., Avantor Performance Materials, Inc. Cat. No. 0616-12, Center Valley, Pa.). In some embodiments, the salivating agent is present in an amount of about 0.1 wt. % to about 1 wt. % (e.g., total weight before casting), such as about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, or about 1 wt. % (e.g., total weight before casting). In some embodiments, the oral thin film comprises substantially no added salivating agent or no added salivating agent.

In some embodiments, the excipient comprises a penetration enhancer. In some embodiments, the penetration enhancer comprises, consists essentially of, or consists of glyceryl monooleate, Tween 80, oleyl alcohol, or a combination thereof. In some embodiments, the penetration enhancer comprises, consists essentially of, or consists of glyceryl monooleate (e.g., Spectrum Chemicals Cat. No. G1017, New Brunswick, N.J.). In some embodiments, the penetration enhancer comprises, consists essentially of, or consists of Tween 80 (e.g., MP Biomedicals Cat, No. 103170, Santa Ana, Calif.). In some embodiments, the penetration enhancer comprises, consists essentially of, or consists of oleyl alcohol (e.g., Super Refined™ Novol NF, Croda International Plc, East Yorkshire, England). In some embodiments, the one or more penetration enhancers is/are present in a total amount of about 1 wt. % to about 15 wt. % (e.g., total weight before casting), such as about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, or about 15 wt. % (e.g., total weight before casting). In some embodiments, the penetration enhancers comprises an combination of two or more penetration enhancers, wherein each penetration enhancer is individually present in an amount of about 1 wt. % to about 15 wt. % (e.g., total weight before casting), such as about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, or about 15 wt. % (e.g., total weight before casting). In some embodiments, the oral thin film comprises substantially no penetration enhancer, or no penetration enhancer.

In some embodiments, an oral thin film consistent with the present disclosure comprises a matrix polymer, about 2 wt. % cannabidiol, about 0.5 wt. % to about 20 wt. % total plasticizer, about 0.2 wt. % to about 2 wt. % flavorant, about 0.1 wt. % to about 1 wt. % salivating agent, about 1 wt. % to about 15 wt. % penetration enhancer, if present, about 0.1 wt. % to about 1 wt. % colorant, about 0.01 wt. % to about 0.5 wt. % preservative, wherein the amounts refer to the weight of each component present in the composition before casting into thin film form.

In some embodiments, an oral thin film consistent with the present disclosure comprises about 5 wt. % to about 7 wt. % cannabidiol, about 20 wt. % to about 22 wt. % matrix polymer, about 0.5 wt. % to about 1 wt. % sweetener, about 8 wt. % to about 12 wt. % plasticizer, about 2 wt. % to about 4 wt. % penetration enhancer, about 0.5 wt. % to about 1 wt. % flavorant, about 0.2 wt. % to about 0.4 wt. % colorant, about 0.2 wt. % to about 0.4 wt. % salivating agent, about 0.05 wt. % to about 0.1 wt. % preservative, wherein the amounts refer to the weight of each component present in the composition after being cast into thin film form (e.g., into a 1 mm-thick thin film).

In some embodiments, an oral thin film consistent with the present disclosure comprises about 6.0 wt. % cannabidiol, about 21.6 wt. % matrix polymer, about 0.7 wt. % sweetener, about 10.8 wt. % plasticizer, about 3.6 wt. % penetration enhancer, about 0.7 wt. % flavorant, about 0.4 wt. % colorant, about 0.4 wt. % salivating agent, and about 0.07 wt. % preservative, wherein the amounts refer to the weight of each component present in the composition after being cast into thin film form (e.g., into a 1 mm-thick thin film).

In some embodiments, an oral thin film consistent with the present disclosure comprises about 6.0 wt. % cannabidiol, about 21.6 wt. % Soluplus matrix polymer, about 0.7 wt. % xylitol, about 10.8 wt. % Tween 80, about 3.6 wt. % propylene glycol, about 0.7 wt. % peppermint oil, about 0.4 wt. % chlorophyll, about 0.4 wt. % citric acid, and about 0.07 wt. % BHT, wherein the amounts refer to the weight of each component present in the composition after being cast into thin film form (e.g., into a 1 mm-thick thin film).

A composition for use in accordance with the disclosure can be formulated as one or more dosage units. The terms “dose unit” and “dosage unit” herein refer to a portion of a pharmaceutical composition that contains an amount of a therapeutic agent (e.g., CBD) suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In some embodiments, compositions of the present disclosure are in the form of orally deliverable dosage forms or units. More specifically, embodiments of the present disclosure include compositions in the form of oral thin films. Such oral thin films provide stable storage of the CBD active agent over time, and convenience in administering an accurate specified dose of the CBD upon administration of a single oral thin film.

In discussing the amount of CBD in a composition of the present disclosure, this may be split over several dosage forms. There is a limit as to the size for oral administration due to practical limits on the size of a single oral thin film that is convenient and tolerable to a subject. An adult subject may be able to tolerate a relatively larger sized oral thin film compared to a child. In addition, CBD is a highly hydrophobic molecule and thus there is a practical limit on the amount of CBD that may be included in an oral thin film of given dimensions. For example and without limitation, if a subject is to be administered 300 mg of CBD per day, the subject may be administered more than one CBD oral thin film dosage form each day, such as three 100-mg CBD oral thin films, or ten 30-mg CBD oral thin films.

In another embodiment, compositions of the present disclosure comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition, and that does not produce unacceptable toxicity or interaction with other components in the composition. By way of example only, a pharmaceutical composition according to the present disclosure may comprise one or more of: antioxidants, surfactants, preservatives, flavoring agents, co-solvents, viscosity aids, suspension aids, and lipophilic phases.

Oral thin films may be cast using any suitable method known in the art. Generally, a viscous solution is prepared comprising cannabidiol (e.g., a cannabis extract) as described herein, and the viscous solution is fed through a casting machine. The extruded film is then dried or heated to evaporate excess solvent, leaving a stable sheet. The sheet may then be divided into dosage units or other sized portions for further processing.

In some embodiments, the viscous solution is degassed (e.g., using a vacuum pump) before casting.

In some embodiments, the present disclosure provides an oral delivery form comprising a cannabis extract. In some embodiments, the cannabis extract comprises cannabidiol. In some embodiments, the cannabis extract comprises at least about 95% cannabidiol. In some embodiments, the cannabis extract comprises at least about 98% cannabidiol. In some embodiments, the cannabis extract comprises at least about 99% cannabidiol. In some embodiments, the oral delivery form is an oral thin film. In some embodiments, the oral thin film is in a unit dose form comprising about 30 mg of the cannabidiol. In some embodiments, the oral thin film is in a unit dose form comprising about 100 mg of the cannabidiol. In some embodiments, the unit dose form comprises a width of about 2.5 cm and/or a length of about 2.5 cm. In some embodiments, the cannabis extract is present in an amount of about 3 wt. % to about 12 wt. %, based on a total weight of the oral delivery form before casting into unit dose form. In some embodiments, the oral thin film further comprises hydroxypropylcellulose. In some embodiments, propylene glycol is present in an amount of about 5 wt. %, based on a total weight of the oral delivery form before casting into unit dose form. In some embodiments, the oral thin film further comprises a polyvinyl caprolactam-polyvinyl acetate-polyethylene glycol graft copolymer. In some embodiments, propylene glycol is present in an amount of about 5 wt. % to about 15 wt. %, based on a total weight of the oral delivery form before casting into unit dose form. In some embodiments, the cannabidiol comprises Δ¹-cannabidiol. In some embodiments, the cannabidiol comprises Δ²-cannabidiol. In some embodiments, the cannabidiol comprises Δ³-cannabidiol. In some embodiments, the cannabidiol comprises Δ^{3,7}-cannabidiol. In some embodiments, the cannabidiol comprises Δ⁴-cannabidiol. In some embodiments, the cannabidiol comprises Δ⁵-cannabidiol. In some embodiments, the cannabidiol comprises Δ⁶-cannabidiol. In some embodiments, the oral delivery form further comprises an excipient. In some embodiments, the excipient comprises a preservative. In some embodiments, the preservative comprises butylated hydroxytoluene. In some embodiments, the excipient comprises a flavoring agent. In some embodiments, the flavoring agent comprises peppermint oil. In some embodiments, the the excipient comprises a coloring agent. In some embodiments, the coloring agent comprises chlorophyll. In some embodiments, the excipient comprises citric acid.

In some embodiments, the present disclosure provides an oral thin film comprising cannabidiol. In some embodiments, the cannabidiol represents at least about 95% of all pharmaceutically active agents present in the oral thin film. In some embodiments, the cannabidiol represents at least about 98% of all pharmaceutically active agents present in the oral thin film. In some embodiments, the cannabidiol represents at least about 99% of all pharmaceutically active agents present in the oral thin film. In some embodiments, the oral thin film is in a unit dose form comprising about 30 mg of the cannabidiol. In some embodiments, the oral thin film is in a unit dose form comprising about 100 mg of the cannabidiol. In some embodiments, the unit dose form comprises a width of about 2.5 cm and/or a length of about 2.5 cm. In some embodiments, the cannabidiol is present in an amount of about 3 wt. % to about 12 w %, based on a total weight of the oral thin film before casting into unit dose form. In some embodiments, the oral thin film further comprises hydroxypropylcellulose. In some embodiments, propylene glycol is present in an amount of about 5 wt. %, based on a total weight of the oral thin film before casting into unit dose form. In some embodiments, the oral thin film further comprises a polyvinyl caprolactam-polyvinyl acetate-polyethylene glycol graft copolymer. In some embodiments, propylene glycol is present in an amount of about 5 wt. % to about 15 wt. %, based on a total weight of the oral thin film before casting into unit dose form. In some embodiments, the cannabidiol comprises Δ¹-cannabidiol. In some embodiments, the cannabidiol comprises Δ²-cannabidiol. In some embodiments, the cannabidiol comprises Δ³-cannabidiol. In some embodiments, the cannabidiol comprises Δ^{3,7}-cannabidiol. In some embodiments, the cannabidiol comprises Δ⁴-cannabidiol. In some embodiments, the cannabidiol comprises Δ⁵-cannabidiol. In some embodiments, the cannabidiol comprises Δ⁶-cannabidiol. In some embodiments, the oral thin film further comprises an excipient. In some embodiments, the excipient comprises a preservative. In some embodiments, the preservative comprises butylated hydroxytoluene. In some embodiments, the excipient comprises a flavoring agent. In some embodiments, the flavoring agent comprises peppermint oil. In some embodiments, the excipient comprises a coloring agent. In some embodiments, the coloring agent comprises chlorophyll. In some embodiments, the excipient comprises citric acid.

### EXAMPLES

### Example 1. General Film-Forming Procedures

A prepared polymer solution of (i) 31 wt. % fungal polysaccharide (Pullulan USP-NF, Hayashibara Co. LTD) in water, (ii) 30 wt. % polyvinyl caprolactam-polyvinyl acetate-polyethylene glycol graft co-polymer (Soluplus, BASF Cat. No. 410343-S) in water, (iii) 17 wt. % pharmaceutical grade hydroxypropyl cellulose-based polymer (Klucel JXF, Ashland Cat. No. 420040) in water, (iv) 23 wt,% pharmaceutical grade hydroxypropylmethyl cellulose-based polymer (Benecel E15 PH, Ashland Cat. No. 794037) in water, or (v) 25 wt. % hypromellose acetate succinate AS-LG (Shin-Etsu Cat. No. AQOAT AS LG) in 1:1 ethanol/water was mixed with an amount of anhydrous ethanol sufficient to disperse the cannabis extract (BioSynthesis Pharma Group Ltd., Sandwich, England: Cat. No, CBD1001, >99.0% CBD, referred to herein as “CBD1001”), a plasticizer, and a salivating agent.

The mixture (optionally degassed) was fed through a motorized film applicator equipped with a square blade applicator having a desired slot size (e.g., 1.27 mm or 50 mil, 2 mm, or 3 mm) onto a glass bed lined with a polyester liner.

The glass bed, polyester liner, and wet film were dried in a forced air oven at 40° C. for about 24 hours. The dried thin film was then peeled from the liner using a narrow-tipped steel spatula.

Compositions of several stable oral thin films produced by this general method are described in more detail in Tables 1-39.

TABLE 1
Pullulan solution concentration (% w/w) = 31.0%
Target batch mass = 3 g

| Chemicals | Weight (%) | Mass (g) | Volume (μL) |
|---|---|---|---|
| Pullulan + Water | 93.6615 | 2.810 | |
| Water | | 1.938 | |
| Pullulan | | 0.872 | |
| Propylene glycol | 1 | 0.030 | 28.85 |
| Peppermint oil | 1 | 0.030 | 33.41 |
| Citric acid | | | |
| CBD1001 | 4.339 | 0.130 | |
| (not included) | | | |
| | 100 | 3.000 | |

CBD1001 concentration in ethanol = 36 mg/mL
Ethanol volume needed = 3.62 mL

TABLE 2
Soluplus solution concentration (% w/w) = 29.8%
Target batch mass = 3 g

| Chemicals | Weight (%) | Mass (g) | Volume (μL) |
|---|---|---|---|
| Soluplus + Water | 93.8135 | 2.814 | |
| Water | | 1.975 | |
| Soluplus | | 0.839 | |
| Propylene glycol | 1 | 0.030 | 28.85 |
| Peppermint oil | 1 | 0.030 | 33.41 |
| Citric acid | | | |
| CBD1001 | 4.187 | 0.126 | |
| (not included) | | | |
| | 100 | 3.000 | |

CBD1001 concentration in ethanol = 36 mg/mL
Ethanol volume needed = 3.49 mL

TABLE 3
Klucel JXF solution concentration (% w/w) = 23.1%
Target batch mass = 3 g

| Chemicals | Weight (%) | Mass (g) | Volume (μL) |
|---|---|---|---|
| Klucel JXF + Water | 94.6697 | 2.840 | |
| Water | | 2.185 | |
| Klucel JXF | | 0.655 | |
| Propylene glycol | 1 | 0.030 | 28.85 |
| Peppermint oil | 1 | 0.030 | 33.41 |
| Citric acid | | | |
| CBD1001 | 3.33 | 0.100 | |
| (not included) | | | |
| | 100 | 3.000 | |

CBD1001 concentration in ethanol = 36 mg/mL
Ethanol volume needed = 2.78 mL

TABLE 4
Benecel E15 solution concentration (% w/w = 17.0%
Target batch mass = 3 g

| Chemicals | Weight (%) | Mass (g) | Volume (μL) |
|---|---|---|---|
| Benecel E15 + Water | 95.4545 | 2.864 | |
| Water | | 2.377 | |
| Benecel E15 | | 0.487 | |
| Propylene glycol | 1 | 0.030 | 28.85 |
| Peppermint oil | 1 | 0.030 | 33.41 |
| Citric acid | | | |
| CBD1001 | 2.545 | 0.076 | |
| (not included) | | | |
| | 100 | 3.000 | |

CBD1001 concentration in ethanol = 36 mg/mL
Ethanol volume needed = 2.12 mL

TABLE 5
Klucel JXF solution concentration (% w/w) = 23.1%
Target batch mass = 3 g
Target CBD1001 = 100 mg

| Chemicals | Weight (%) | Mass (g) | Volume (μL) |
|---|---|---|---|
| Klucel JXF + Water | 94.6697 | 2.840 | |
| Water | | 2.185 | |
| Klucel JXF | | 0.655 | |
| Propylene glycol | 1 | 0.030 | 28.85 |
| Peppermint oil | 1 | 0.030 | 33.41 |
| Citric acid | | | |
| CBD1001 | 3.330 | 0.100 | |
| (not included) | | | |
| | 100 | 3.000 | |

CBD1001 concentration in ethanol = 36 mg/mL

TABLE 6
Klucel JXF solution concentration (% w/w) = 23.1%
Target batch mass = 3 g
Target CBD1001 = 100 mg

| Chemicals | Weight (%) | Mass (g) | Volume (μL) |
|---|---|---|---|
| Klucel JXF + Water | 94.6697 | 2.840 | |
| Water | | 2.185 | |
| Klucel JXF | | 0.655 | |
| Propylene glycol | 1 | 0.030 | 28.85 |
| Peppermint oil | 1 | 0.030 | 33.41 |
| Citric acid | | | |
| CBD1001 | 3.330 | 0.100 | |
| | 100 | 3.000 | |

CBD1001 concentration in ethanol = 36 mg/mL
Ethanol volume needed = 2.775 mL

TABLE 7
Klucel JXF solution concentration (% w/w) = 23.1%
Target batch mass = 4 g
Target CBD1001 = 100 mg

| Chemicals | Weight (%) | Mass (g) | Volume (μL) |
|---|---|---|---|
| Klucel JXF + Water | 94.6697 | 3.790 | |
| Water | | 2.913 | |
| Klucel JXF | | 0.874 | |
| Propylene glycol | 1 | 0.040 | 38.46 |
| Peppermint oil | 1 | 0.040 | 44.35 |
| Citric acid | | | |
| CBD1001 | 3.330 | 0.133 | |
| | 100 | 4.000 | |

CBD1001 concentration in ethanol = 36 mg/mL
Ethanol volume needed = 3.70 mL

TABLE 8
Klucel JXF solution concentration (% w/w) = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 94.4313 | 2.833 | |
| Water | | 2.179 | |
| Klucel JXF | | 0.654 | |
| Propylene glycol | 1 | 0.030 | 28.85 |
| Peppermint oil | 1 | 0.030 | 33.41 |
| Citric acid | | | |
| CBD1001 | 3.569 | 0.107 | |
| | 100 | 3.000 | |

CBD1001 concentration in ethanol = 36 mg/mL
Ethanol volume needed = 2.974 mL

TABLE 9
Klucel JXF solution concentration (% w/w) = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 94.4313 | 2.833 | |
| Water | | 2.179 | |
| Klucel JXF | | 0.654 | |
| Propylene glycol | 1 | 0.030 | 28.85 |
| Peppermint oil | 1 | 0.030 | 33.41 |
| Citric acid | | | |
| CBD1001 | 3.569 | 0.107 | |
| | 100 | 3.000 | |

CBD1001 concentration in ethanol = 50 mg/mL
Ethanol volume needed = 2.141 mL

TABLE 10
Klucel JXF solution concentration (% w/w) = 23.1%
Target batch mass = 3 g
Target CBD1001 = 100 mg; wt % = 50%
Target film thickness = 0.270 mm

| Chemicals | Weight (%) | Mass g | Volume (μL) |
|---|---|---|---|
| Klucel JXF + Water | 86.9655 | 2.609 | |
| Water | | 2.007 | |
| Klucel JXF | | 0.602 | |
| Propylene glycol | 1 | 0.030 | 28.85 |
| Peppermint oil | 1 | 0.030 | 33.41 |
| Citric acid | | | |
| CBD1001 | 11.034 | 0.331 | |
| | 100 | 3.000 | |

CBD1001 concentration in ethanol = 150 mg/mL
Ethanol volume needed = 2.207 mL

TABLE 11
Klucel JXF solution concentration (% w/w) = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 93.3197 | 2.800 | |
| Water | | 2.154 | |
| Klucel JXF | | 0.646 | |
| Propylene glycol | 2 | 0.060 | 57.69 |
| Peppermint oil | 1 | 0.030 | 33.41 |
| Citric acid | | | |
| CBD1001 | 3.680 | 0.110 | |
| (not included) | | | |
| | 100 | 3.000 | |

CBD1001 concentration in ethanol = 200 mg/mL
Ethanol volume needed = 0.552 mL

TABLE 12
Klucel JXF solution concentration (% w/w) = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 89.985 | 2.700 | |
| Water | | 2.077 | |
| Klucel JXF | | 0.623 | |
| Propylene glycol | 5 | 0.150 | 144.23 |
| Peppermint oil | 1 | 0.030 | 33.41 |
| Citric acid | | | |
| CBD1001 | 4.015 | 0.120 | |
| (not included) | | | |
| | 100 | 3.000 | |

CBD1001 concentration in ethanol = 200 mg/mL
Ethanol volume needed = 0.602 mL

TABLE 13
Klucel JXF solution concentration (% w/w) = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 84.8275 | 2.533 | |
| Water | | 1.948 | |
| Klucel JXF | | 0.584 | |
| Propylene glycol | 10 | 0.300 | 288.46 |
| Peppermint oil | 1 | 0.030 | 33.41 |
| Citric acid | | | |
| CBD1001 | 4.572 | 0.137 | |
| (not included) | | | |
| | 100 | 3.000 | |

CBD1001 concentration in ethanol = 200 mg/mL
Ethanol volume needed = 0.686 mL

TABLE 14
Klucel JXF solution concentration (% w/w) = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 89.985 | 2.700 | |
| Water | | 2.077 | |
| Klucel JXF | | 0.623 | |
| Propylene glycol | 5 | 0.150 | 144.23 |
| Peppermint oil | 1 | 0.030 | 33.41 |
| Citric acid | | | |
| CBD1001 | 4.015 | 0.120 | |
| | 100 | 3.000 | |

CBD1001 concentration in ethanol = 200 mg/mL
Ethanol volume needed = 1.251 mL
Stock volume needed = 0.602 mL
Final CBD1001 concentration = 65.0 mg/, L
Final volume needed = 1.853 mL

TABLE 15
Klucel JXF solution concentration % w/w = 23.1%
Target batch mass = 3 g
Target CBD1001 = 30 mg; wt % = 15%
Target film thickness = 0.270 mm

| Chemicals | Weight (%) | Mass (g) | Volume (μL) |
|---|---|---|---|
| Klucel JXF + Water | 89.985 | 2.700 | |
| Water | | 2.077 | |
| Klucel JXF | | 0.623 | |
| Propylene glycol | 5 | 0.150 | 144.23 |
| Peppermint oil | 1 | 0.030 | 33.41 |
| Citric acid | | | |
| CBD1001 | 4.015 | 0.120 | |
| | 100 | 3.000 | |

CBD1001 concentration in ethanol = 200 mg/mL
Ethanol volume needed = 1.807 mL
Stock volume needed = 0.602 mL
Final CBD1001 concentration = 50.0 mg/, L
Final volume needed = 2.409 mL

TABLE 16
Klucel JXF solution concentration (% w/w) = 23.1%
Target batch mass = 3 g
Target CBD1001 = 30 mg; wt % = 10%
Target film thickness = 0.270 mm

| Chemicals | Weight (%) | Mass (g) | Volume (μL) |
|---|---|---|---|
| Klucel JXF + Water | 88.725 | 2.662 | |
| Water | | 2.048 | |
| Klucel JXF | | 0.614 | |
| Propylene glycol | 5 | 0.150 | 144.23 |
| Peppermint oil | 1 | 0.030 | 33.41 |
| Chlorophyll | 0.5 | 0.015 | 15.00 |
| Citric acid | 0.5 | 0.015 | |
| Xylitol | 1 | 0.03 | |
| (not included) | | | |
| BHT | 0.1 | 0.003 | |
| CBD1001 | 3.18 | 0.095 | |
| | 100 | 3.000 | |

Ethanol volume needed (36 mg/mL) = 2.65 mL

TABLE 17
Klucel JXF solution concentration (% w/w) = 23.1%
Target batch mass = 3 g
Target CBD1001 (wt %) = 40%

| Chemicals | Weight (%) | Mass (g) | Volume (μL) |
|---|---|---|---|
| Klucel JXF + Water | 74.9665 | 2.249 | |
| Water | | 1.730 | |
| Klucel JXF | | 0.519 | |
| Propylene glycol | 5 | 0.150 | 144.23 |
| Peppermint oil | 1 | 0.030 | 33.41 |
| Chlorophyll | 0.5 | 0.015 | 15.00 |
| Citric acid | 0.5 | 0.015 | |
| Xylitol | 1 | 0.03 | |
| (not included) | | | |
| BHT | 0.1 | 0.003 | |
| CBD1001 | 16.93 | 0.508 | |
| | 100 | 3.000 | |

CBD1001 concentration in ethanol = 290 mg/mL
Ethanol volume needed = 1.75 mL

TABLE 18
Soluplus solution concentration (% w/w) = 44.0%
Target batch mass = 3 g
Target TA-001-201 = 30 mg; wt % = 20%
Wet film thickness = 2.0 mm

| Chemicals | Weight (%) | Mass (g) | Volume (μL) |
|---|---|---|---|
| Soluplus + Water | 80.9685 | 2.429 | |
| Water | | 1.360 | |
| Soluplus | | 1.069 | |
| Propylene glycol | 5 | 0.150 | 144.23 |
| Peppermint oil | 1 | 0.030 | 33.26 |
| Chlorophyll | 0.5 | 0.015 | 15.00 |
| Citric acid | 0.5 | 0.015 | |
| Xylitol | 1 | 0.03 | |
| (not included) | | | |
| BHT | 0.1 | 0.003 | |
| CBD1001 | 10.93 | 0.328 | |
| (not included) | | | |
| | 100 | 3.000 | |

CBD1001 concentration in ethanol = 825 mg/mL
Ethanol volume needed = 0.40 mL

TABLE 19
Soluplus solution concentration (% w/w) = 44.0%
Target batch mass = 3 g
Target TA-001-201 = 30 mg; wt % = 20%
Wet film thickness = 2.0 mm

| Chemicals | Weight (%) | Mass (g) | Volume (μL) |
|---|---|---|---|
| Soluplus + Water | 75.3380 | 2.260 | |
| Water | | 1.266 | |
| Soluplus | | 0.994 | |
| Propylene glycol | 10 | 0.300 | 288.46 |
| Peppermint oil | 1 | 0.030 | 33.26 |
| Chlorophyll | 0.5 | 0.015 | 15.00 |
| Citric acid | 0.5 | 0.015 | |
| Xylitol | 1 | 0.03 | |
| (not included) | | | |
| BHT | 0.1 | 0.003 | |
| CBD1001 | 11.56 | 0.347 | |
| (not included) | | | |
| | 100 | 3.000 | |

CBD1001 concentration in ethanol = 870 mg/mL
Ethanol volume needed = 0.40 mL

TABLE 20
Soluplus solution concentration (% w/w) = 44.0%
Target batch mass = 3 g
Target TA-001-201 = 30 mg; wt % = 20%
Wet film thickness = 2.0 mm

| Chemicals | Weight (%) | Mass (g) | Volume (μL) |
|---|---|---|---|
| Soluplus + Water | 64.0765 | 1.922 | |
| Water | | 1.076 | |
| Soluplus | | 0.846 | |
| Propylene glycol | 20 | 0.600 | 576.92 |
| Peppermint oil | 1 | 0.030 | 33.26 |
| Chlorophyll | 0.5 | 0.015 | 15.00 |
| Citric acid | 0.5 | 0.015 | |
| Xylitol | 1 | 0.03 | |
| (not included) | | | |
| BHT | 0.1 | 0.003 | |
| CBD1001 | 12.82 | 0.385 | |
| (not included) | | | |
| | 100 | 3.000 | |

CBD1001 concentration in ethanol = 960 mg/mL
Ethanol volume needed = 0.40 mL

TABLE 21
Soluplus solution concentration (% w/w) = 44.0%
Target batch mass = 5 g
Target TA-001-201 = 30 mg; wt % = 4.5%
Wet film thickness = 3.0 mm

| Chemicals | Weight (%) | Mass (g) | Volume (μL) |
|---|---|---|---|
| Soluplus + Water | 89.6595 | 4.483 | |
| Water | | 2.510 | |
| Soluplus | | 1.973 | |
| Propylene glycol | 5 | 0.250 | 240.38 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.24 | 0.112 | |
| | 100 | 5.000 | |

Ethanol volume needed = 0.30 mL

TABLE 22
Soluplus solution concentration (% w/w) = 44.0%
Target batch mass = 5 g
Target TA-001-201 = 30 mg; wt % = 4.5%
Wet film thickness = 3.0 mm

| Chemicals | Weight (%) | Mass (g) | Volume (μL) |
|---|---|---|---|
| Soluplus + Water | 84.5302 | 4.227 | |
| Water | | 2.367 | |
| Soluplus | | 1.860 | |
| Propylene glycol | 10 | 0.500 | 480.77 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.37 | 0.118 | |
| | 100 | 5.000 | |

Ethanol volume needed = 0.30 mL

TABLE 23
Soluplus solution concentration (% w/w) = 44.0%
Target batch mass = 4 g
Target TA-001-201 = 30 mg; wt % = 6.5%
Wet film thickness = 2.0 mm

| Chemicals | Weight (%) | Mass (g) | Volume (μL) |
|---|---|---|---|
| Soluplus + Water | 83.437 | 3.337 | |
| Water | | 1.869 | |
| Soluplus | | 1.468 | |
| Propylene glycol | 10 | 0.400 | 384.62 |
| Peppermint oil | 1 | 0.040 | 44.35 |
| Chlorophyll | 0.5 | 0.020 | 20.00 |
| Citric acid | 0.5 | 0.020 | |
| Xylitol | 1 | 0.040 | |
| (not included) | | | |
| BHT | 0.1 | 0.004 | |
| CBD1001 | 3.46 | 0.139 | |
| | 100 | 4.000 | |

Ethanol volume needed = 0.30 mL

TABLE 24
Soluplus solution concentration (% w/w) = 44.0%
Target batch mass = 4 g
Target TA-001-201 = 30 mg; wt % = 6.5%
Wet film thickness = 2.0 mm

| Chemicals | Weight (%) | Mass (g) | Volume (μL) |
|---|---|---|---|
| Soluplus + Water | 78.2484 | 3.130 | |
| Water | | 1.753 | |
| Soluplus | | 1.377 | |
| Propylene glycol | 15 | 0.600 | 576.92 |
| Peppermint oil | 1 | 0.040 | 44.35 |
| Chlorophyll | 0.5 | 0.020 | 20.00 |
| Citric acid | 0.5 | 0.020 | |
| Xylitol | 1 | 0.040 | |
| (not included) | | | |
| BHT | 0.1 | 0.004 | |
| CBD1001 | 3.65 | 0.146 | |
| | 100 | 4.000 | |

Ethanol volume needed = 0.30 mL

TABLE 25
Soluplus solution concentration (% w/w) = 44.0%
Target batch mass = 5 g
Target TA-001-201 = 30 mg; wt % = 4.5%
Wet film thickness = 3.0 mm

| Chemicals | Weight (%) | Mass (g) | Volume (μL) |
|---|---|---|---|
| Soluplus + Water | 74.2716 | 3.714 | |
| Water | | 2.080 | |
| Soluplus | | 1.634 | |
| Propylene glycol | 10 | 0.500 | 480.77 |
| Oleyl alcohol | 10 | 0.500 | 588.93 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.63 | 0.131 | |
| | 100 | 5.000 | |

Ethanol volume needed = 1.00 mL

TABLE 26
Soluplus solution concentration (% w/w) = 44.0%
Target batch mass = 5 g
Target TA-001-201 = 30 mg; wt % = 4.5%
Wet film thickness = 3.0 mm

| Chemicals | Weight (%) | Mass (g) | Volume (μL) |
|---|---|---|---|
| Soluplus + Water | 74.2716 | 3.714 | |
| Water | | 2.080 | |
| Soluplus | | 1.634 | |
| Propylene glycol | 10 | 0.500 | 480.77 |
| Tween 80 | 10 | 0.500 | 462.96 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.63 | 0.131 | |
| | 100 | 5.000 | |

Ethanol volume needed = 1.00 mL

TABLE 27
Soluplus solution concentration (% w/w) = 44.0%
Target batch mass = 5 g
Target TA-001-201 (wt %) = 4.5%

| Chemicals | Weight (%) | Mass (g) | Volume (μL) |
|---|---|---|---|
| Soluplus + Water | 74.2716 | 3.714 | |
| Water | | 2.080 | |
| Soluplus | | 1.634 | |
| Propylene glycol | 10 | 0.500 | 480.77 |
| Glyceryl monooleate | 10 | 0.500 | |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.63 | 0.131 | |
| | 100 | 5.000 | |

Ethanol volume needed = 1.00 mL

TABLE 28
Soluplus solution concentation (% w/w) = 44.0%
Target batch mass = 5 g
Target TA-001-201 = 30 mg; (wt %) = 4.5%
Wet film thickness = 3 mm

| Chemicals | Weight (%) | Mass (g) | Volume (μL) |
|---|---|---|---|
| Soluplus + Water | 79.401 | 3.970 | |
| Water | | 2.223 | |
| Soluplus | | 1.747 | |
| Propylene glycol | 5 | 0.250 | 240.38 |
| Oleyl alcohol | 10 | 0.500 | 588.93 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.50 | 0.125 | |
| | 100 | 5.000 | |

Ethanol volume needed = 1.30 mL

TABLE 29
Soluplus solution concentration (% w/w) = 44.0%
Target batch mass = 5 g
Target TA-001-201 = 30 mg; (wt %) = 4.5%
Wet film thickness = 3 mm

| Chemicals | Weight (%) | Mass (g) | Volume (μL) |
|---|---|---|---|
| Soluplus + Water | 79.401 | 3.970 | |
| Water | | 2.223 | |
| Soluplus | | 1.747 | |
| Propylene glycol | 5 | 0.250 | 240.38 |
| Tween 80 | 10 | 0.500 | 462.96 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.50 | 0.125 | |
| | 100 | 5.000 | |

Ethanol volume needed = 1.30 mL

TABLE 30
Soluplus solution concentration (% w/w) = 44.0%
Target batch mass = 5 g
Target TA-001-201 = 30 mg; (wt %) = 4.5%
Wet film thickness = 3 mm

| Chemicals | Weight (%) | Mass (g) | Volume (μL) |
|---|---|---|---|
| Soluplus + Water | 79.401 | 3.970 | |
| Water | | 2.223 | |
| Soluplus | | 1.747 | |
| Propylene glycol | 5 | 0.250 | 240.38 |
| Glyceryl monooleate | 10 | 0.500 | |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.50 | 0.125 | |
| | 100 | 5.000 | |

Ethanol volume needed = 1.30 mL

TABLE 31
Soluplus solution concentration (% w/w) = 44.0%
Target batch mass = 5 g
Target TA-001-201 = 30 mg; (wt %) = 4.5%
Wet film thickness = 3 mm

| Chemicals | Weight (%) | Mass (g) | Volume (μL) |
|---|---|---|---|
| Soluplus + Water | 79.401 | 3.970 | |
| Water | | 2.223 | |
| Soluplus | | 1.747 | |
| Propylene glycol | 5 | 0.250 | 240.38 |
| Glyceryl monooleate | 3.33 | 0.167 | |
| Tween 80 | 3.33 | 0.167 | 154.32 |
| Oleyl alcohol | 3.33 | 0.167 | 196.31 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.50 | 0.125 | |
| | 100 | 5.000 | |

Ethanol volume needed = 1.20 mL

TABLE 32
Soluplus solution concentation (% w/w) = 44.0%
Target batch mass = 5 g
Target TA-001-201 = 30 mg; (wt %) = 4.5%
Wet film thickness = 3 mm

| Chemicals | Weight (%) | Mass (g) | Volume (μL) |
|---|---|---|---|
| Soluplus + Water | 79.401 | 3.970 | |
| Water | | 2.223 | |
| Soluplus | | 1.747 | |
| Propylene glycol | 10 | 0.500 | 480.77 |
| Tween 80 | 5 | 0.250 | 231.48 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 2.50 | 0.125 | |
| | 100 | 5.000 | |

Ethanol volume needed = 1.30 mL

TABLE 33
Soluplus solution concentration (% w/w) = 44.0%
Target batch mass = 5 g
Target TA-001-201 = 30 mg; (wt %) = 13.5%
Wet film thickness = 1 mm

| Chemicals | Weight (%) | Mass (g) | Volume (μL) |
|---|---|---|---|
| Soluplus + Water | 73.994 | 3.700 | |
| Water | | 2.072 | |
| Soluplus | | 1.628 | |
| Propylene glycol | 10 | 0.500 | 480.77 |
| Oleyl alcohol | 5 | 0.250 | 294.46 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 7.91 | 0.395 | |
| | 100 | 5.000 | |

Ethanol volume needed = 1.20 mL
Volume added to Soluplus = 1.761 mL

TABLE 34
Soluplus solution concentration (% w/w) = 44.0%
Target batch mass = 5 g
Target TA-001-201 = 30 mg; (wt %) = 13.5%
Wet film thickness = 1 mm

| Chemicals | Weight (%) | Mass (g) | Volume (μL) |
|---|---|---|---|
| Soluplus + Water | 73.994 | 3.700 | |
| Water | | 2.072 | |
| Soluplus | | 1.628 | |
| Propylene glycol | 10 | 0.500 | 480.77 |
| Tween 80 | 5 | 0.250 | 231.48 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 7.91 | 0.395 | |
| | 100 | 5.000 | |

Ethanol volume needed = 1.20 mL
Volume added to Soluplus = 1.761 mL

TABLE 35
Soluplus solution concentration (% w/w) = 44.0%
Target batch mass = 5 g
Target TA-001-201 = 30 mg; (wt %) = 13.5%
Wet film thickness = 1 mm

| Chemicals | Weight (%) | Mass (g) | Volume (μL) |
|---|---|---|---|
| Soluplus + Water | 73.994 | 3.700 | |
| Water | | 2.072 | |
| Soluplus | | 1.628 | |
| Propylene glycol | 10 | 0.500 | 480.77 |
| Glyceryl monooleate | 5 | 0.250 | |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 7.91 | 0.395 | |
| | 100 | 5.000 | |

Ethanol volume needed = 1.20 mL
Volume added to Soluplus = 1.761 mL

TABLE 36
Soluplus solution concentation (% w/w) = 44.0%
Target batch mass = 5 g
Target TA-001-201 = 30 mg; (wt %) = 13.5%
Wet film thickness = 1 mm

| Chemicals | Weight (%) | Mass (g) | Volume (μL) |
|---|---|---|---|
| Soluplus + Water | 73.994 | 3.700 | |
| Water | | 2.072 | |
| Soluplus | | 1.628 | |
| Propylene glycol | 10 | 0.500 | 480.77 |
| Tween 80 | 5 | 0.250 | 231.48 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 7.91 | 0.395 | |
| | 100 | 5.000 | |

Ethanol volume needed = 1.20 mL

TABLE 37
Soluplus solution concentration (% w/w) = 44.0%
Target batch mass = 5 g
Target TA-001-201 = 30 mg; (wt %) = 13.5%
Wet film thickness = 1 mm

| Chemicals | Weight (%) | Mass (g) | Volume (μL) |
|---|---|---|---|
| Soluplus + Water | 71.8304 | 3.592 | |
| Water | | 2.011 | |
| Soluplus | | 1.580 | |
| Propylene glycol | 12 | 0.600 | 576.92 |
| Tween 80 | 5 | 0.250 | 231.48 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 8.07 | 0.403 | |
| | 100 | 5.000 | |

Ethanol volume needed = 1.05 mL

TABLE 38
Soluplus solution concentration (% w/w) = 44.0%
Target batch mass = 5 g
Target TA-001-201 = 30 mg; (wt %) = 13.5%
Wet film thickness = 1 mm

| Chemicals | Weight (%) | Mass (g) | Volume (μL) |
|---|---|---|---|
| Soluplus + Water | 68.585 | 3.429 | |
| Water | | 1.920 | |
| Soluplus | | 1.509 | |
| Propylene glycol | 15 | 0.750 | 721.15 |
| Tween 80 | 5 | 0.250 | 231.48 |
| Peppermint oil | 1 | 0.050 | 55.43 |
| Chlorophyll | 0.5 | 0.025 | 25.00 |
| Citric acid | 0.5 | 0.025 | |
| Xylitol | 1 | 0.050 | |
| (not included) | | | |
| BHT | 0.1 | 0.005 | |
| CBD1001 | 8.31 | 0.416 | |
| | 100 | 5.000 | |

Ethanol volume needed = 0.90 mL

TABLE 39

| Ingredient | % w/w |
|---|---|
| Soluplus | 21.640 |
| Water | 27.542 |
| Xylitol | 0.717 |
| Propylene glycol | 10.757 |
| Tween 80 | 3.586 |
| Peppermint Oil | 0.717 |
| Chlorophyll | 0.359 |
| Citric Acid | 0.359 |
| BHT | 0.072 |
| CBD1001 | 5.963 |
| Ethanol | 28.290 |
| Total | 100 |

TABLE 40
Test Formulas.

| | | Cannabis | | | | | | | Film | |
|---|---|---|---|---|---|---|---|---|---|---|
| Test | | Extract | | | | | | | thickness | |
| Formula | | (>99.0% | | | Salivating | Penetration | | | (at | Batch |
| No. | Polymer | Cannabidiol | Plasticizer | Flavorant | Agent | Enhancer | Colorant | Preservative | casting) | size |
| 1 | Klucel | 10 wt. % | 5 wt. % | 1 wt. % | 0.5 wt. % | — | 0.5 | 0.1 wt. % | 3 mm | 3 g |
| | JXF | | PG | PO | CA | | wt. % C | BHT | | |
| 2 | Klucel | 30 mg | 5 wt. % | 1 wt. % | 0.5 wt. % | — | 0.5 | 0.1 wt. % | 1 mm | 3 g |
| | JXF | equivalent | PG | PO | CA | | wt. % C | BHT | | |
| | | in 140 | | | | | | | | |
| | | mg/mL | | | | | | | | |
| | | ethanol | | | | | | | | |
| 3 | Klucel | 30 mg | 5 wt. % | 1 wt. % | 0.5 wt. % | — | 0.5 | 0.1 wt. % | 0.75 mm | 3 g |
| | JXF | equivalent | PG | PO | CA | | wt. % C | BHT | | |
| | | in 290 | | | | | | | | |
| | | mg/mL | | | | | | | | |
| | | ethanol | | | | | | | | |
| 4 | Solupl | 30 mg in | 10 wt. % | 1 wt. % | 0.5 wt. % | — | 0.5 | 0.1 wt. % | 2 mm | 4 g |
| | us | ethanol | PG | PO | CA | | wt. % C | BHT | | |
| 5 | Solupl | 30 mg in | 15 wt. % | 1 wt. % | 0.5 wt. % | — | 0.5 | 0.1 wt. % | 2 mm | 4 g |
| | us | ethanol | PG | PO | CA | | wt. % C | BHT | | |
| 6 | Solupl | 30 mg in | 10 wt. % | 1 wt. % | 0.5 wt. % | — | 0.5 | 0.1 wt. % | 3 mm | 5 g |
| | us | ethanol | PG | PO | CA | | wt. % C | BHT | | |
| 7 | Solupl | 4.5 wt. % in | 5 wt. % | 1 wt. % | 0.5 wt. % | 10 wt. % | 0.5 | 0.1 wt. % | 3 mm | 5 g |
| | us | ethanol | PG | PO | CA | Tween 80 | wt. % C | BHT | | |
| 8 | Solupl | 4.5 wt. % in | 5 wt. % | 1 wt. % | 0.5 wt. % | 10 wt. % | 0.5 | 0.1 wt. % | 3 mm | 5 g |
| | us | ethanol | PG | PO | CA | GM | wt. % C | BHT | | |
| 9 | Solupl | 4.5 wt. % in | 5 wt. % | 1 wt. % | 0.5 wt. % | 10 wt. % | 0.5 | 0.1 wt. % | 3 mm | 5 g |
| | us | ethanol | PG | PO | CA | OA | wt. % C | BHT | | |
| 10 | Solupl | 4.5 wt. % in | 10 wt. % | 1 wt. % | 0.5 wt. % | 5 wt. % | 0.5 | 0.1 wt. % | 3 mm | 5 g |
| | us | ethanol | PG | PO | CA | Tween 80 | wt. % C | BHT | | |
| 11 | Solupl | 4.5 wt. % in | 5 wt. % | 1 wt. % | 0.5 wt. % | 3.33 wt. % | 0.5 | 0.1 wt. % | 3 mm | 5 g |
| | us | ethanol | PG | PO | CA | Tween 80 | wt. % C | BHT | | |
| | | | | | | 3.33 wt. % | | | | |
| | | | | | | OA | | | | |
| | | | | | | 3.33 wt. % | | | | |
| | | | | | | GM | | | | |
| 12 | Solupl | 13.5 wt. % | 10 wt. % | 1 wt. % | 0.5 wt. % | 5 wt. % | 0.5 | 0.1 wt. % | 1 mm | 5 g |
| | us | in ethanol | PG | PO | CA | OA | wt. % C | BHT | | |
| 13 | Solupl | 13.5 wt. % | 10 wt. % | 1 wt. % | 0.5 wt. % | 5 wt. % | 0.5 | 0.1 wt. % | 1 mm | 5 g |
| | us | in ethanol | PG | PO | CA | Tween 80 | wt. % C | BHT | | |
| 14 | Solupl | 13.5 wt. % | 10 wt. % | 1 wt. % | 0.5 wt. % | 5 wt. % | 0.5 | 0.1 wt. % | 1 mm | 5g |
| | us | in ethanol | PG | PO | CA | GM | wt. % C | BHT | | |
| 15 | Solupl | 30 mg in | 5 wt. % | 1 wt. % | 0.5 wt. % | — | 0.5 | 0.1 wt. % | 3 mm | 5 g |
| | us | ethanol | PG | PO | CA | | wt. % C | BHT | | |
| 16 | Solupl | 4.5 wt. % in | 10 wt. % | 1 wt. % | 0.5 wt. % | 10 wt. % | 0.5 | 0.1 wt. % | 3 mm | 5 g |
| | us | ethanol | PG | PO | CA | Tween 80 | wt. % C | BHT | | |
| 17 | Solupl | 4.5 wt. % in | 10 wt. % | 1 wt. % | 0.5 wt. % | 10 wt. % | 0.5 | 0.1 wt. % | 3 mm | 5 g |
| | us | ethanol | PG | PO | CA | GM | wt. % C | BHT | | |
| 18 | Solupl | 4.5 wt. % in | 10 wt. % | 1 wt. % | 0.5 wt. % | 10 wt. % | 0.5 | 0.1 wt. % | 3 mm | 5 g |
| | us | ethanol | PG | PO | CA | OA | wt. % C | BHT | | |
| 19 | Solupl | 13.5 wt. % | 10 wt. % | 1 wt. % | 0.5 wt. % | 5 wt. % | 0.5 | 0.1 wt. % | 1 mm | 5g |
| | us | in ethanol | PG | PO | CA | Tween 80 | wt. % C | BHT | | |
| 20 | Solupl | 13.5 wt. % | 12 wt. % | 1 wt. % | 0.5 wt. % | 5 wt. % | 0.5 | 0.1 wt. % | 1 mm | 5 g |
| | us | in ethanol | PG | PO | CA | Tween 80 | wt. % C | BHT | | |
| 21 | Solupl | 13.5 wt. % | 15 wt. % | 1 wt. % | 0.5 wt. % | 5 wt. % | 0.5 | 0.1 wt. % | 1 mm | 5 g |
| | us | in ethanol | PG | PO | CA | Tween 80 | wt. % C | BHT | | |
| 22 | Klucel | 30 mg in | 5 wt. % | 1 wt. % | — | — | — | — | 3 mm | 3 g |
| | JXF | 50 mg/mL | PG | PO | | | | | | |
| | | ethanol | | | | | | | | |
| 23 | Klucel | 30 mg in | 5 wt. % | 1 wt. % | 0.5 wt. % | — | — | 0.1 wt. % | 3 mm | 3 g |
| | JXF | ethanol | PG | PO | CA | | | BHT | | |
| 24 | Klucel | 30 mg in | 5 wt. % | 1 wt. % | — | — | — | — | 3 mm | 3 g |
| | JXF | 200 | PG | PO | | | | | | |
| | | mg/mL | | | | | | | | |
| | | ethanol | | | | | | | | |
| | | and 50 | | | | | | | | |
| | | mg/mL | | | | | | | | |
| | | water | | | | | | | | |
| 25 | Klucel | 30 mg in | 5 wt. % | 1 wt. % | — | — | — | — | 3 mm | 3 g |
| | JXF | 65 mg/mL | PG | PO | | | | | | |
| | | ethanol | | | | | | | | |
| 26 | Klucel | 10 wt. % | 5 wt. % | 1 wt. % | 0.5 wt. % | — | 0.5 | 0.1 wt. % | 3 mm | 3 g |
| | JXF | | PG | PO | CA | | wt. % C | BHT | | |
| 27 | Bence | 100 mg in | 1 wt. % | 1 wt. % | — | — | — | — | 3 mm | 3 g |
| | el E15 | ethanol | PG | PO | | | | | | |
| | PH | | | | | | | | | |
| 28 | Klucel | 30 mg in | 5 wt. % | 1 wt. % | — | — | — | — | 3 mm | 3 g |
| | JXF | 200 | PG | PO | | | | | | |
| | | mg/mL | | | | | | | | |
| | | ethanol | | | | | | | | |
| 29 | Klucel | 30 mg in | 5 wt. % | 1 wt. % | — | — | — | — | 3 mm | 3 g |
| | JXF | 100 | PG | PO | | | | | | |
| | | mg/mL | | | | | | | | |
| | | ethanol | | | | | | | | |
| 30 | Klucel | 100 mg in | 1 wt. % | 1 wt. % | — | — | — | — | 3 mm | 3 g |
| | JXF | 150 | PG | PO | | | | | | |
| | | mg/mL | | | | | | | | |
| | | ethanol | | | | | | | | |
| 31 | Klucel | 100 mg in | 1 wt. % | 1 wt. % | — | — | — | — | 3 mm | 3 g |
| | JXF | ethanol | PG | PO | | | | | | |
| 32 | Klucel | 30 mg in | 10 wt. % | 1 wt. % | — | — | — | — | 3 mm | 3 g |
| | JXF | ethanol | PG | PO | | | | | | |
| 33 | Klucel | 30 mg in | 1 wt. % | 1 wt. % | — | — | — | — | 3 mm | 3 g |
| | JXF | 36 mg/mL | PG | PO | | | | | | |
| | | ethanol | | | | | | | | |
| 34 | Klucel | 30 mg in | 2 wt. % | 1 wt. % | — | — | — | — | 3 mm | 3 g |
| | JXF | ethanol | PG | PO | | | | | | |
| 35 | Klucel | 30 mg in | 5 wt. % | 1 wt. % | — | — | — | — | 3 mm | 3 g |
| | JXF | ethanol | PG | PO | | | | | | |
| 36 | Klucel | 100 mg in | 1 wt. % | 1 wt. % | — | — | — | — | — | 4 g |
| | JXF | ethanol | PG | PO | | | | | | |
| 37 | Klucel | 100 mg in | 1 wt. % | 1 wt. % | — | — | — | — | 1.27 mm | 3 g |
| | JXF | ethanol | PG | PO | | | | | wet- | |
| | | | | | | | | | coat | |
| 38 | Klucel | 100 mg in | 1 wt. % | 1 wt. % | — | — | — | — | — | 3 g |
| | JXF | ethanol | PG | PO | | | | | | |

Table 40 Abbreviations:
PG: propylene glycol
PO: peppermint oil
CA: citric acid
GM: glyceryl monooleate
C: chlorophyll
BHT: butylated hydroxytoluene
OA: oleyl alcohol

### Example 2. Tissue Permeation

Two 1×1 cm² square pieces of Formulation 21 of Example 1 were cut, weighed and dissolved in MeOH (at ˜2 mg/mL). The films dissolved quickly (<3.0 minutes) and completely in the MeOH. A 0.221 mg/mL working solution (WS) of each was prepared by dilution into 50:50 MeoH:H2O, followed by further dilution to a 5 μg/mL test solution. Duplicate 100 μL injections of the nominal 5 μg/mL test solutions were performed. HPLC was conducted on a guard-protected Waters XBridge C18 (5 μm; 4.6×250 mm) analytical column eluted with 35:65 0.1% TFA (pH 6 with 5% ACN):ACN at a 1.5 mL/min flow rate. Eluant was monitored for absorbance at 230 nm. Preparations were compared to freshly prepared 5 μg/mL TA-001-2015 calibrator. Results are shown in Table 41.

TABLE 41
Gravimetric Analysis of Cannabidiol Thin Film

| | | | | Back | |
|---|---|---|---|---|---|
| | | Gravimetric | Back Calculated | Calculated | |
| | Mass of | Total | Cannabidiol | Cannabidiol | |
| Cannabidiol Oral | Film Piece | Mass/Volume | Content | in Film Piece | Cannabidiol |
| Thin Film | (mg) | (mg/mL) | (mg/mL) | (mg) | Loading (%) |
| Formulation #21-a | 30.25 | 11.88 | 1.89 | 4.82 | 15.94 |
| Formulation #21-b | 29.63 | 11.65 | 1.83 | 4.65 | 15.69 |

| | | | |
|---|---|---|---|
| avg | 29.94 | avg | 15.81 |
| % RSD | 1.46 | % RSD | 1.09 |

A PermeGear flow-through diffusion cell system with membrane supports (In-Line, Hellertown, Pa.) was assembled using purified water (NANOpure® DIamond™ Life Science (UV/UF) ultrapure water, Barnstead International, Dubuque, Iowa) with 10% ethanol filtered through a 0.2 μm nylon membrane filter was used as the receiver fluid.

Tissue harvested from Yucatan miniature pigs (Sinclair Bio Resources, Auxvasse, Mo.) was transported on wet ice and stored at −20° C. until used. Immediately before use, the top layers of the tissue were removed with a dermatome. A second cutting with the dermatome was used for the permeation studies.

Briefly, a 4.84 cm² section of tissue was arranged in the diffusion cell such that the permeation area of the tissue was 0.95 cm². Diffusion cells were kept at 32° C. with a circulating water bath. Flow rate was set at about 0.5 rpm. Each cell was charged with a 0.9525 cm² circular disc of the test thin film of Test Formulas 1-14 and 19-21 of Example 1 by first applying 10 μL of nanopure water to the tissue. After applying the thin film to the wet tissue, an additional 50 μL of nanopure water was applied on top of the thin film to simulate exposure to saliva. The samples were then applied to the equilibrated permeation cells and sealed with a stopper. For some studies, a polypropylene surgical mesh (PPKM603, Textile Development Associates Inc. Brookfield, Conn.) overlay was used to ensure the thin film had secure contact to the tissue sample.

Samples were collected at 1 hour, 2 hours, 3, hours, 4, hours, 5, hours, 6, hours, 10, hours, 14 hours, and 18 hours for initial studies; additional samples were collected at 2 hours, 4 hours, 6 hours, 8 hours, 10 hours, 12 hours, 16 hours, 20 hours, and 24 hours for some studies. All samples were stored at 4° C. until analyzed by HPLC.

Following the diffusion experiment, the test film was removed from each tissue sample. Tissue was washed with nanopure water, dried and tape-stripped twice with book tape to remove any surface drug. The diffusional area was excised, chopped and placed into a pre-weighed vial. After recording the tissue weight, 10 mL of acetonitrile was added and the vials sealed. After shaking overnight at room temperature, the liquid portions were analyzed by HPLC.

Quantification of cannabidiol was performed by high performance liquid chromatography (HPLC) using a Waters 2695 Alliance Separations Module and column heater with a Waters 2487 Dual Wavelength Absorbance Detector. The solvent system consisted of 20% 0.1% TFA pH=3.0 and 80% acetonitrile and was run through a Waters XBridge™ 5 μm, 4.6 mm i.d.×250 mm column at a flow rate of 1.5 mL/min. Samples were injected in duplicate at 100 μL each. The limit of quantification was 0.5 μg/mL of cannabidiol. Samples were assayed the day of collection or within 48 hours after collection. When peaks from the original cannabis extract interfered with test analytes, the isocratic elution was changed to 35% 0.1% TFA buffer:65% acetonitrile and the run time was extended to 30 minutes from 6 minutes.

Results are shown in Table 42 below.

TABLE 42
Permeation Study Results.

| | | | Tissue |
|---|---|---|---|
| | | Cumulative amount¹ | concentrations |
| Formulation | n | (nmol) | (μmol/g) |
| 1 | 3 | 1.76 ± 1.75 | 2.09 ± 0.97 |
| 2 | 3 | 3.05 ± 1.98 | 3.19 ± 1.20 |
| 3 | 3 | 1.69 ± 0.90 | 1.98 ± 0.17 |
| 4 | 3 | TA² | 5.34 ± 3.30 |
| 2 | 3 | TA² | 1.13 ± 0.07 |
| 4 | 3 | TA² | 0.75 ± 0.15 |
| 5 | 3 | TA² | 1.53 ± 1.35 |
| 6 | 3 | TA² | 1.76 ± 0.64 |
| 2 | 1 | TA² | 1.19 |
| 4 | 1 | TA² | 0.51 |
| 7 | 3 | TA² | 0.19 ± 0.05 |
| 8 | 3 | 1.78 (n = 1) | 0.23 ± 0.03 |
| 9 | 3 | 1.09, 0.24 (n = 2) | 0.09 ± 0.01 |
| 10 | 3 | TA² | 0.35 ± 0.06 |
| 11 | 3 | TA² | 0.31 ± 0.17 |
| 4 | 1 | 0.47 | 0.86 |
| 7 | 1 | 0.60 | 0.33 |
| 10 | 1 | 0.50 | 0.93 |
| 12 | 3 | TA² | 1.53 ± 1.36 |
| 13 | 3 | 51.72 ± 36.45 | 0.48 ± 0.16 |
| 14 | 3 | TA² | 1.05 ± 1.60 |
| 13 | 3 | 3.67 ± 2.11 | 0.78 ± 0.06 |
| 19 | 3 | 1.38 ± 0.83 | 0.42 ± 0.22 |
| 20 | 3 | 10.65 ± 4.46 | 0.37 ± 0.12 |
| 21 | 3 | 22.09 ± 29.84 | 0.58 ± 0.06 |
| 21 | 6 | NA³ | 0.28 ± 0.09 |
| 21 | 4 | 15.07 ± 2.62 | 0.93 ± 0.31 |

²TA = trace amounts, below LOQ
³NA = due to limited volume of samples and assay interference, weren't able to reanalyze samples

### Example 3: Stability and Disintegration of Oral Films

TA-001 oral films were prepared as described above and stored at 38° C., 75% relative humidity for up to 28 days. The temperature was selected so that the films would not undergo significant melting during the studies, since it was observed that higher temperatures can result in unsuitable softening of the films. On days 0, 3, 7, 14, 21 and 28, six TA-001 oral films (2.5×2.5 cm; ˜17,244% drug load based on Day 0 analysis of films) were examined for color, clarity, thickness and mass prior to being dissolved in methanol (nominal 7.5 mg total film weight/mL). These methanolic stock solutions were weighed then immediately used to prepare diluted solutions (8 μL QS to 2 mL with 50:50 H₂O:methanol; ˜5.17 μg TA-001/mL) for analysis by HPLC-UV. Samples were injected (100 μL duplicates) onto a guard-protected (5μ; 4.6×10 mm) Waters X-Bridge C18 (5μ; 4.6×250 mm at 35° C.) analytical column and eluted at 10.1 min with a gradient mixture of mobile phase A (0.1% trifluoroacetic acid, pH 3, containing 5% acetonitrile) and B (100% acetonitrile) delivered at 1.5 mL/min, and monitored for absorbance at 230 nm. Gradient conditions were 35% mobile phase A:65% mobile phase B for 12 min, followed by a linear ramp to 100% mobile phase B for 30 min, constant at 100% mobile phase B for 10 min, returning linearly to 35% mobile phase A:65% mobile phase B over 5 min with a final equilibration at 35:65 mobile phase A:mobile phase B of 5 min (62 min total run time). Peak areas from duplicate injections were averaged and divided by the gravimetrically determined concentration of TA-001 in the injection solutions to determine the response ratio (RR) for analyte in each oral film. Stability of TA-001 in the oral films under the specified storage conditions was calculated by the change in RRs as compared to the initial Day 0 RRs. Results from the 28 day accelerated stability studies are shown below in Tables 43 and 44. Based on the results at 28 days, there was no apparent degradation of the active ingredient in the TA-001 oral films throughout the study.

For the disintegration studies, placebo films were placed into ˜21 mL of isotonic phosphate buffer, pH 6.8 to determine how long the films would take to dissolve. On Day 0, it took 12.7±6.0 min (n=3) for the films to fully disintegrate in vitro. At Day 7, it took 13-14 min for the films to disintegrate. At Day 14, it took 10-11 min for the films to disintegrate.

TABLE 43
Degradation Study Results

| | | Film | Color | | | | |
|---|---|---|---|---|---|---|---|
| | | Thickness | Change | Crystallization | Film | Extract | % Loading (by |
| | Replicate | mm | Yes or No | Yes or No | Mass mg | Content mg | weight) |
| 0 day | 1 | 0.23 | No | No | 160.72 | 27.087 | 16.9 |
| | 2 | 0.23 | No | No | 151.40 | 26.659 | 17.6 |
| | 3 | 0.26 | No | No | 148.06 | 25.001 | 16.9 |
| | 4 | 0.22 | No | No | 154.01 | 27.335 | 17.7 |
| | 5 | 0.24 | No | No | 177.36 | 31.388 | 17.7 |
| | 6 | 0.23 | No | No | 164.25 | 29.163 | 17.8 |
| 3 | 1 | 0.27 | No | No | 164.91 | 29.681 | 18.0 |
| days | 2 | 0.28 | No | No | 154.45 | 27.249 | 17.6 |
| | 3 | 0.25 | No | No | 157.06 | 27.682 | 17.6 |
| | 4 | 0.24 | No | No | 177.08 | 32.080 | 18.1 |
| | 5 | 0.24 | No | No | 152.5 | 27.335 | 17.9 |
| | 6 | 0.25 | No | No | 162.54 | 29.065 | 17.9 |
| 7 | 1 | 0.24 | No | No | 156.84 | 26.514 | 16.9 |
| days | 2 | 0.31 | No | No | 11987 | 20.602 | 17.2 |
| | 3 | 0.26 | No | No | 140.66 | 25.082 | 17.8 |
| | 4 | 0.25 | No | No | 152.88 | 26.580 | 17.4 |
| | 5 | 0.23 | No | No | 158.53 | 27.826 | 17.6 |
| | 6 | 0.24 | No | No | 148.23 | 25.930 | 17.5 |
| 14 | 1 | 0.25 | No | No | 157.63 | 26.801 | 17.0 |
| days | 2 | 0.22 | No | No | 148.52 | 26.920 | 18.1 |
| | 3 | Could not | No | No | 136.09 | 24.343 | 17.9 |
| | | determine | | | | | |
| | 4 | 0.24 | No | No | 151.05 | 26.206 | 17.3 |
| | 5 | 0.25 | No | No | 140.47 | 24.704 | 17.6 |
| | 6 | 0.26 | No | No | 150.74 | 25.597 | 17.0 |
| 21 | 1 | 0.22 | No | No | 142.89 | 25.202 | 17.6 |
| days | 2 | 0.23 | No | No | 122.56 | 21.651 | 17.7 |
| | 3 | 0.25 | No | No | 146.37 | 26.267 | 17.9 |
| | 4 | 0.27 | No | No | 143.32 | 25.618 | 17.9 |
| | 5 | 0.26 | No | No | 162.02 _ | 28.835 | 17.8 |
| | 6 | 0.27 | No | No | 140.94 | 25.025 | 17.8 |
| 28 | 1 | 0.25 | No | No | 128.22 | 21.991 | 17.2 |
| days | 2 | 0.23 | No | No | 141.48 | 24.909 | 17.6 |
| | 3 | 0.26 | No | No | 150.83 | 26.380 | 17.5 |
| | 4 | 0.24 | No | No | 164.5 | 28.479 | 17.3 |
| | 5 | 0.26 | No | No | 179.81 | 31.698 | 17.6 |
| | 6 | 0.24 | No | No | 147.88 | 26.696 | 18.1 |

TABLE 44
Degradation Study Results

| | Response Ratio (Peak | Stability | |
|---|---|---|---|
| | Area/TA001 Conc.; | (Percent Day 0 | |
| Day | Avg, n = 6) | Avg. Response Ratio) | % RSD |
| 0 | 125,325 | 100.0 | 2.5 |
| 3 | 128,489 | 102.5 | 1.1 |
| 7 | 125,094 | 99.8 | 1.8 |
| 14 | 125,785 | 100.4 | 2.7 |
| 21 | 127,875 | 102.0 | 0.67 |
| 28 | 126,153 | 100.7 | 1.8 |

| | |
|---|---|
| 0.05, 0.1, 0.5, 1, 5, 10 μg/mL | 126,862; 1.55%; 6 |
| Calibrator Response Ratios | |
| (Avg; % RSD; n) | |

From the foregoing, it will be appreciated that specific embodiments of the invention have been described herein for purposes of illustration, but that various modifications may be made without deviating from the scope of the invention. Accordingly, the invention is not limited except as by the appended claims.

1. An oral dosage form comprising a plant extract.

2. The oral dosage form of claim 1, wherein the plant extract comprises cannabidiol.

3. The oral dosage form of any one preceding claim, wherein the plant extract comprises at least about 95% cannabidiol.

4. The oral dosage form of any one preceding claim, wherein the plant extract comprises at least about 98% cannabidiol.

5. The oral dosage form of any one preceding claim, wherein the plant extract comprises at least about 99% cannabidiol.

6. The oral dosage form of any one preceding claim, wherein the oral dosage form is an oral thin film.

7. The oral dosage form of claim 6 or claim 7, wherein the oral thin film is in a unit dose form comprising about 30 mg of the cannabidiol.

8. The oral dosage form of claim 6 or claim 7, wherein the oral thin film is in a unit dose form comprising about 100 mg of the cannabidiol.

9. The oral dosage form of claim 7 or claim 8, wherein the unit dose form comprises a width of about 2.5 cm and/or a length of about 2.5 cm.

10. The oral dosage form of any one preceding claim, wherein the plant extract is present in an amount of about 3 wt. % to about 12 wt. %, based on a total weight of the oral dosage form before casting into unit dose form.

11. The oral dosage form of any one of claims 6 to 10, wherein the oral thin film further comprises hydroxypropylcellulose.

12. The oral dosage form of claim 11, wherein propylene glycol is present in an amount of about 5 wt. %, based on a total weight of the oral dosage form before casting into unit dose form.

13. The oral dosage form of any one of claims 6 to 10, wherein the oral thin film further comprises a polyvinyl caprolactam-polyvinyl acetate-polyethylene glycol graft copolymer.

14. The oral dosage form of claim 13, wherein propylene glycol is present in an amount of about 5 wt. % to about 15 wt. %, based on a total weight of the oral dosage form before casting into unit dose form.

15. The oral dosage form of any one preceding claim, wherein the cannabidiol comprises Δ¹-cannabidiol.

16. The oral dosage form of any one preceding claim, wherein the cannabidiol comprises Δ²-cannabidiol.

17. The oral dosage form of any one preceding claim, wherein the cannabidiol comprises Δ³-cannabidiol.

18. The oral dosage form of any one preceding claim, wherein the cannabidiol comprises Δ^{3,7}-cannabidiol.

19. The oral dosage form of any one preceding claim, wherein the cannabidiol comprises Δ⁴-cannabidiol.

20. The oral dosage form of any one preceding claim, wherein the cannabidiol comprises Δ⁵-cannabidiol.

21. The oral dosage form of any one preceding claim, wherein the cannabidiol comprises Δ⁶-cannabidiol.

22. The oral dosage form of any one preceding claim further comprising an excipient.

23. The oral dosage form of claim 22, wherein the excipient comprises a preservative.

24. The oral dosage form of claim 23, wherein the preservative comprises butylated hydroxytoluene.

25. The oral dosage form of any one of claims 22 to 24, wherein the excipient comprises a flavoring agent.

26. The oral dosage form of claim 25, wherein the flavoring agent comprises peppermint oil.

27. The oral dosage form of any one of claims 22 to 26, wherein the excipient comprises a coloring agent.

28. The oral dosage form of claim 27, wherein the coloring agent comprises chlorophyll.

29. The oral dosage form of any one of claims 22 to 28, wherein the excipient comprises citric acid.

30. An oral thin film comprising cannabidiol.

31. The oral thin film of claim 30, wherein the cannabidiol represents at least about 95% of all pharmaceutically active agents present in the oral thin film.

32. The oral thin film of claim 30 or claim 31, wherein the cannabidiol represents at least about 98% of all pharmaceutically active agents present in the oral thin film.

33. The oral thin film of any one of claims 30 to 32, wherein the cannabidiol represents at least about 99% of all pharmaceutically active agents present in the oral thin film.

34. The oral thin film of any one of claims 30 to 33, wherein the oral thin film is in a unit dose form comprising about 30 mg of the cannabidiol.

35. The oral thin film of any one of claims 30 to 34, wherein the oral thin film is in a unit dose form comprising about 100 mg of the cannabidiol.

36. The oral thin film of claim 34 or claim 35, wherein the unit dose form comprises a width of about 2.5 cm and/or a length of about 2.5 cm.

37. The oral thin film of any one preceding claim, wherein the cannabidiol is present in an amount of about 3 wt. % to about 12 wt. %, based on a total weight of the oral thin film before casting into unit dose form.

38. The oral thin film of any one of claims 30 to 37, wherein the oral thin film further comprises hydroxypropylcellulose.

39. The oral thin film of claim 38, wherein propylene glycol is present in an amount of about 5 wt. %, based on a total weight of the oral thin film before casting into unit dose form.

40. The oral delivery form of any one of claims 30 to 37, wherein the oral thin film further comprises a polyvinyl caprolactam-polyvinyl acetate-polyethylene glycol graft copolymer.

41. The oral thin film of claim 40, wherein propylene glycol is present in an amount of about 5 wt. % to about 15 wt. %, based on a total weight of the oral thin film before casting into unit dose form.

42. The oral thin film of any one of claims 30 to 41, wherein the cannabidiol comprises Δ¹-cannabidiol.

43. The oral thin film of any one of claims 30 to 42, wherein the cannabidiol comprises Δ²-cannabidiol.

44. The oral thin film of any one of claims 30 to 43, wherein the cannabidiol comprises Δ³-cannabidiol.

45. The oral thin film of any one of claims 30 to 44, wherein the cannabidiol comprises Δ^{3,7}-cannabidiol.

46. The oral thin film of any one of claims 30 to 45, wherein the cannabidiol comprises Δ⁴-cannabidiol.

47. The oral thin film of any one of claims 30 to 46, wherein the cannabidiol comprises Δ⁵-cannabidiol.

48. The oral thin film of any one of claims 30 to 47, wherein the cannabidiol comprises Δ⁶-cannabidiol.

49. The oral thin film of any one of claims 30 to 48 further comprising an excipient.

50. The oral thin film of claim 49, wherein the excipient comprises a preservative.

51. The oral thin film of claim 50, wherein the preservative comprises butylated hydroxytoluene.

52. The oral thin film of any one of claims 49 to 51, wherein the excipient comprises a flavoring agent.

53. The oral thin film of claim 52, wherein the flavoring agent comprises peppermint oil.

54. The oral thin film of any one of claims 49 to 53, wherein the excipient comprises a coloring agent.

55. The oral thin film of claim 54, wherein the coloring agent comprises chlorophyll.

56. The oral thin film of any one of claims 49 to 55, wherein the excipient comprises citric acid.
